(19) United States
(12) Patent Application Publication
(10) Pub. No.: US 2009/0298158 A1
(43) Pub. Date: Dec. 3, 2009

(54) ADIABATIC COMPACTION OF ALGAE BIOMASS FOR EXTRACTION OF BIOFUEL

(76) Inventors: Glenn Thomas, East Amherst, NY (US); Lennart Lindell, Dekalb, IL (US)

Correspondence Address: WALTER W. DUFT;LAW OFFICES OF WALTER W. DUFT, 8616 MAIN ST, SUITE 2, WILLIAMSVILLE, NY, 14221, US

(21) Appl. No.: 12/467,262

(22) Filed: May 16, 2009

Related U.S. Application Data
(60) Provisional application No. 61/054,135, filed on May 17, 2008.

(51) Int. Cl. C12N 1/12

(52) U.S. Cl. 435/257.1

(57) ABSTRACT

A technique for extracting biofuel from algae biomass using high velocity adiabatic impact compaction, comprising impacting a quantity of algae biomass with a power ram at a controlled velocity to deliver an impulse of sufficient magnitude to disrupt the outer cell wall structure.

### CROSS-REFERENCE TO RELATED APPLICATIONS

This application claims the benefit of U.S. Provisional Patent Application Ser. No. 61/054,135, filed on May 17, 2008 (the '135 application). The contents of the '135 application are hereby incorporated by this reference in their entirety as if fully set forth herein.

### BACKGROUND

1. Field of the Invention

The present invention generally relates to the recovery of biofuel from biomass. More specifically, the invention concerns oil extraction from algae.

2. Description of the Prior Art

Bioorganic fuels (biofuels) obtained from living or recently dead biological material (biomass) are currently being investigated as part of mankind's quest to develop alternatives to petrochemical and other fossil fuel energy sources. Much attention has been focused on biofuels acquired from terrestrial sources, particularly, human-consumable grains such as corn, legumes and other field crops, as well as a variety of non-human-consumable plant species, including grasses, wood, stalks and crop waste. Comparatively little research has been done in the area of aquatic biofuels. Of these, algae is particularly promising due to its high yield per unit area. An acre of algae produces 7-30 times more energy than the highest yielding land-based plant species, due in large part to its comparatively faster growth cycle. Research suggests that algae could supply enough fuel to meet all of America's transportation needs in the form of biodiesel using as little as 0.2% of the nation's land. In particular, enough algae can be grown to replace all transportation fuels in the U.S. on only 15,000 square miles, or 9.6 million acres of land. This is approximately the size of the State of Maryland.

Techniques currently used to extract biofuel from algae include treatment with chemical solvents, enzymatic extraction, ultrasonic extraction, CO₂ extraction, osmotic shock, isostatic mechanical pressing (direct extraction), and isostatic pressing in combination with chemical solvent extraction. Each of these techniques seeks to degrade or disrupt the algae cell wall structure, so that the lipids present within the cell interior may be released and recovered. All are relatively expensive and time consuming. Direct extraction appears to hold promise from a cost standpoint, and produces higher grades of fuel than chemical extraction methods. However, the technique is slow and relatively inefficient due to the resiliency of the algae cell wall structure. This is why mechanical pressing is typically employed in combination with a chemical solvent treatment.

It is to improvements in the extraction of biofuel from algae that the present invention is directed. In particular, an alternative form of direct extraction is proposed that can be used to recovery biofuel at high yields without supplemental chemical solvent processing.

### SUMMARY

A technique is provided for extracting biofuel from algae biomass using high velocity adiabatic impact compaction. According to example embodiments herein, a quantity of algae biomass can be impacted with a power ram at a controlled velocity to deliver an impulse of sufficient magnitude to disrupt the outer cell wall structure, allowing the internal oils within the algae cellular environment to be expelled and recovered.

### BRIEF DESCRIPTION OF THE DRAWINGS

The foregoing and other features and advantages of the invention will be apparent from the following more particular description of example embodiments, as illustrated in the accompanying drawing, in which:

FIG. 1 is a cross-sectional centerline view of an example high velocity adiabatic impact compaction apparatus that may be used for biofuel extraction from algae in accordance with the detailed description presented below.

### DETAILED DESCRIPTION OF EXAMPLE EMBODIMENTS

Applicants have discovered that a mechanical pressing technique known as high velocity adiabatic impact (HVAI) compaction may be used to efficiently recover biofuel from algae. HVAI compaction is a metal forming technique whereby high energy is delivered very rapidly to a metal target using a pneumatically-driven, spring-driven or hydraulically-driven power ram without significant heat transfer to the target or the tooling. The technique is well suited for applications such as precision cut-off, zero clearance blanking, and net shape forming. U.S. Pat. Nos. 3,956,953, 4,245,493, 4,470,330, 6,571,596, disclosing adiabatic impact press apparatus, are illustrative. The contents of these patents are hereby incorporated herein by this reference in their entirety.

HVAI compaction has also been shown to be viable when used in combination with sintering and/or precompaction for adiabatic coalescence of powder metal to form articles of high relative density. Densification is achieved by intensive shock waves delivered by the high speed ram traveling at velocities ranging from 2-10 meters/second (with higher speeds up to 30 meters per second also being mentioned in the literature).

In experiments conducted by applicants, HVAI compaction was used to crack a quantity of semi-dried powdered algae, thereby facilitating the release and extraction of internal oils from the interior cellular environment. The algae was a microalgae salt water species having approximately 40% lipid content by weight, but it is anticipated that the present HVAI technique may also be used with other microalgae species and also with macroalgae species. The present compaction technique was implemented by placing a dried algae biomass sample in a die and impacting it with a power ram (of any suitable type) at a controlled velocity to deliver an impulse of sufficient magnitude to disrupt the outer cell wall structure. The power ram velocity and the impulse delivered by the power ram were varied according to the mass of the algae. The mass of the algae samples ranged from approximately 2-14 grams. Impulse represents the change in momentum (I=mΔv) of the power ram as it impacts the algae sample. The quantity “m” represents the mass of the power ram and the quantity “Δv” represents the change in ram velocity resulting from the impact. Because the final ram velocity is zero, the quantity “Δv” (vᵢₙᵢₜᵢₐₗ−v_{final}) becomes the ram velocity immediately prior to impact. It will be appreciated that the impulse “I” with respect to time (mΔv/Δt) provides a measure of the force (F=ma) imparted by the power ram to the algae. Everything else being equal, a sharp impact producing rapid power ram deceleration tends to increase the force, and visa versa.

The power ram mass was approximately 5.6 kg for all test runs. The impulse level was varied by adjusting the pre-impact power ram velocity (by setting the stroke height of the power ram). The pre-impact power ram velocities ranged from approximately 10-50 meters/second. As stated, the post-impact velocity was zero. The impacts lasted less than approximately 3 milliseconds and were thus of very short duration (resulting in large forces). The impulses delivered by the power ram ranged from approximately 55-280 Newton-seconds. These impulse values can be normalized by converting them to specific impulse values, each of which represents the impulse delivered by the power ram divided by the mass of the algae sample. The specific impulse values used in applicants' experiments ranged from approximately 9-24 Newton-seconds/gram of material to be compacted. The kinetic energy (½ mv²) delivered to the algae samples ranged from approximately 280-7100 Joules. The specific kinetic energy levels (kinetic energy per algae sample mass) ranged from approximately 120-503 Joules/gram of material to be compacted. The density of the algae following compaction was approximately 0.7-0.95 grams per cubic centimeter or higher.

It is expected that further experimentation will reveal other power ram mass values, power ram velocities, impulse values, specific impulse values, kinetic energies and specific kinetic energies that are outside the ranges tested by applicants, but which can be used to achieve biofuel extraction from algae. For example, HVAI compaction by delivering an impulse from the power ram as small as approximately 0.5-2.0 Newton-seconds (or lower) for very small algae sample sizes (e.g. as small as approximately 0.1 gram or less) to 5,000-12,000 Newton-seconds (or higher) for very large sample sizes (e.g., as large as approximately 7 kg or more), would be possible. A minimum power ram velocity of 3-5 meters per second (or lower) and a maximum power ram velocity of 150-200 meters per second (or higher) may be appropriate depending on the algae sample size. A minimum power ram velocity of approximately 8 meters per second is expected for algae sample sizes on the order of those tested by applicants (i.e., approximately 2-14 grams). The minimum kinetic energy delivered by the power ram could be as low as 25 joules (or lower). The maximum kinetic energy delivered by the power ram could be as high as 400-1,400 Kilojoules (or greater). Further experimentation may be performed to determine how parameters such as the power ram velocity and impulse can be varied according to the number of impacts (if multiple impacts are used—see below) and the mass of the algae, the type of algae, the algae water content, and the pre-compaction density of the algae. In some cases, the algae biomass may include two or more algae compositions of different mass, type of algae, water content and/or pre-compaction density.

Notwithstanding the foregoing, applicants' experimental data revealed that the oil content per gram of algae, based on qualitative examination, was comparable over a range of power ram velocities, impulses, specific impulses, kinetic energies and specific kinetic energies. This suggests that there may be thresholds of applied power ram velocity, impulse, specific impulse, kinetic energy and specific kinetic energy that will produce maximal oil extraction, above which no further practical benefit is achieved. For example, although the smallest specific impulse used during applicants' testing was approximately 10 Newton-seconds/gram, a threshold specific impulse level that is lower than this may be shown to be effective. Increasing the force of the power ram above the required threshold may be unnecessary and may have the undesired effect of increasing the temperature of the algae/algae oil to an undesirable level. It is known, for example, that increasing the algae/algae oil temperature to above 177° Celsius is detrimental to obtaining the highest energy density and desirable long molecular chain oils from the extraction process. Preferably, the algae oil temperature will be kept below approximately 138° Celsius throughout the HVAI compaction process.

If desired, the HVAI compaction process may be varied to achieve additional beneficial results. For example, an additional ram power stroke may be applied following the initial controlled impact and prior to ejection of the compacted algae biomass. This additional ram power stroke, which may have a duration of up to approximately 100 milliseconds or more, could be used to maximize the algae cell wall disruption. Another option would be to employ one or more additional processing operations. These operations could include, but are not limited to, (1) pre-compaction of the algae biomass (e.g., to a density of between approximately 0.1 to 0.5 grams per cubic centimeter), (2) rapid pressure release post adiabatic impact and/or an additional power stroke to create a cavitation or even explosive effect that promotes oil flow from the compacted algae biomass, (3) post adiabatic impact iso-static pressing of the algae biomass (with or without the use of dies), and (4) post adiabatic impact centrifugal processing in order to aid the extraction of internal oils from the algae biomass, and to separate these oils from remaining algae cell components. A further variation would be to deliver multiple controlled power ram impacts of selective intensity. Multiple power ram impacts in controlled incremental stages followed by one or more additional ram power strokes of selective intensity prior to ejection of the compacted algae biomass could also be used.

The mass of the algae biomass that can be compacted in a single HVAI cycle will depend on the size of the adiabatic press machine being used. Typically, it is expected that each algae sample will have a mass within a range of approximately 0.1-7 kg (although these are by no means the only possible algae sample sizes). Even with relatively small sample sizes, it is anticipated that commercial biofuel production levels can be obtained due to the rapidity of each compaction cycle (e.g., on the order of milliseconds). Moreover, algae sample sizes of greater than 7 kg would be possible if sufficiently large power ram equipment is built.

### Experimental Setup and Test Results

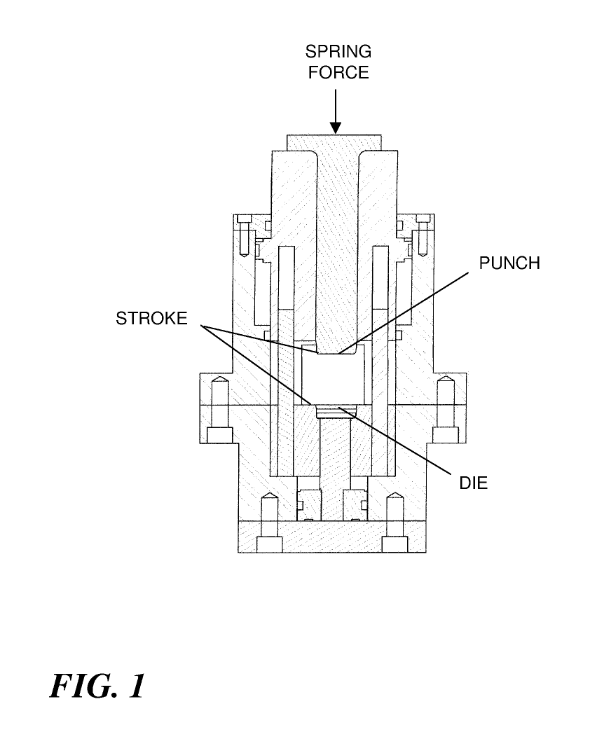

Test machine: Model PIP 100 adiabatic press, built by LMC Inc (DeKalb, Ill.); incorporates vertical ram design. See FIG. 1.

Die & punch design: 1.5 inch diameter circular die with mating spring-driven punch ram having effective mass of approximately 5.6 kg. See FIG. 1.

Algae Material Semi-dried brown salt water microalgae with a 40% lipid content by weight, supplied by Unified Fuels, Mobile, Ala.

Algae information: Approximately 98% or more of water moisture content was removed; consistency of algae was powder-like, looked similar to coarse sand or powder; algae felt dry to the touch, no moisture derived by touch or feel when holding algae. Color in dry state was medium—golden brown.

Test Procedure:
1. Select and adjust machine ram speed at impact by adjusting and setting stoke engagement of the ram prior to impact.
2. Weigh a given amount of algae (ceramic bowl used to hold weighed algae).
3. Deliver algae into pre-located die on machine platen using funnel to drop algae into die.
4. Align die containing algae by centering it under the ram.
5. Release ram; single impact and compaction of algae in die.
6. Remove compacted algae and oil from die and place in plastic sealable bottle, plastic material selected to be inert to algae (polycarbonate/Lexan bottle used). A plastic spatula and sharp metal scalpel knife are tools used to manually remove the algae.
7. Repeat steps 2 through 6 until desired mass of cracked algae is collected.
8. Clean off die when test is completed.

### Experiment #1

PIP 100 velocity at impact: 10 meters/second

| Run # | Algae mass at impact |
|---|---|
| 1 | 2.35 grams |

Observations from Experiment #1 test results included:
1. Oil is clearly visible, and can feel oil released from algae with touch;
2. Algae changed color to darker brown;
3. Oil from algae was ubiquitous: in the algae (broke compacted algae “cake” by hand), and on the metal surfaces of the die.

### Experiment #2

PIP 100 velocity at impact: 25 meters/second

| | | Cumulative sample |
|---|---|---|
| | Algae mass at | mass of impacted |
| Run # | impact, grams | algae, grams |
| 1 | 11.9 (est) | 11.9 |
| 2 | 10.0 (est) | 21.9 |
| 3 | 14.61 | 36.51 |
| 4 | 13.0 | 49.51 |
| 5 | 14.2 | 63.71 |
| 6 | 12.5 (est) | 76.21 |
| 7 | 13.2 | 89.41 |
| 8 | 13.5 | 102.91 |
| 9 | 13.3 | 116.21 |
| 10 | 13.8 | 130.01 |
| 11 | 13.3 | 143.31 |
| 12 | 13.6 | 156.91 |
| 13 | 13.8 | 170.71 |
| 14 | 14.0 | 184.71 |
| 15 | 13.9 | 198.61 |
| 16 | 13.9 | 212.51 |
| 17 | 14.0 | 226.51 |
| 18 | 14.0 | 240.51 |

Completion of Test - approximately 0.24 kilograms of algae cracked/compacted

Observations from Experiment #2 test results:
1. Oil is clearly visible, and can feel oil released from algae with touch;
2. Algae changed color to darker brown; and
3. Oil from algae was ubiquitous: in the algae (broke compacted algae “cake” by hand), and on the metal surfaces of the die.

### Experiment #3

PIP 100 velocity at impact: 25 meters/second

| | | Cumulative sample |
|---|---|---|
| | Algae mass at | mass of impacted |
| Run # | impact, grams | algae, grams |
| 1 | 14.00 | 14.00 |
| 2 | 14.01 | 28.01 |
| 3 | 13.89 | 41.90 |
| 4 | 13.91 | 55.81 |
| 5 | Removed as sample for show | |
| 6 | 13.92 | 69.73 |
| 7 | 13.93 | 83.66 |
| 8 | 13.90 | 97.56 |
| 9 | 13.85 | 111.41 |
| 10 | 14.00 | 125.41 |
| 11 | 13.88 | 139.29 |
| 12 | 14.03 | 153.32 |
| 13 | 13.93 | 167.25 |
| 14 | 14.00 | 181.25 |
| 15 | 13.88 | 195.13 |
| 16 | 13.96 | 209.09 |
| 17 | 13.98 | 223.07 |
| 18 | 13.95 | 237.02 |
| 19 * | 14.03 | 251.05 |

Completion of Test - approximately 0.25 kilograms of algae cracked/compacted
* Test run # 19 done with more velocity: approximately 50 meters/second

Observations from Experiment #3 test results:
1. Oil is definitely visible, and can feel oil released from algae with touch;
2. Algae changed color to darker brown;
3. Oil from algae was ubiquitous: in the algae (broke compacted algae “cake” by hand), and on the metal surfaces of the die;
4. Test run # 19: same result with cracked/compacted algae as earlier runs; nothing noticeably different in color of algae or look, feel and amount of oil present;
5. The total volume of algae oil extracted from algae masses of Experiments #2 and #3 was approximately 225 milliliters.

### Calculation of Impulses, Kinetic Energies, Specific Impulses and Specific Kinetic Energies for Experiments #1-3

PIP 100 Machine Set Points From Test, Impulses and Kinetic Energies:

| Velocity of Ram at | Impulse Delivered at | Kinetic Energy at |
|---|---|---|
| Impact (meters/sec) | Impact (N-sec) | Impact (J) |
| 10 | 55.79 | 282 |
| 25 | 139.48 | 1,763 |
| 50 | 278.96 | 7,051 |

Specific Impulse, from tests:

| Velocity of | Average mass | Specific | Range of Specific |
|---|---|---|---|
| Ram at Impact | of algae/range | Impulse | Impulse |
| (meters/sec) | of mass, grams | (N-sec/gram) | (N-sec/gm) |
| 10 | 2.35 | 23.74 | 23.74 |
| 25 | 13.65/10.0-14.61 | 10.22 | 9.55-13.95 |
| 50 | 14.03 | 19.88 | 19.88 |

Specific Kinetic Energy, from tests:

| Velocity of | Average mass | Specific Kinetic | Range of Specific |
|---|---|---|---|
| Ram at Impact | of algae/range | Energy | Kinetic Energy |
| (meters/sec) | of mass, grams | (J/gram) | (J/gm) |
| 10 | 2.35 | 120.00 | 120.00 |
| 25 | 13.65/10.0-14.61 | 129.16 | 120.67-176.30 |
| 50 | 14.03 | 502.57 | 502.57 |

Accordingly, a technique for adiabatic compaction of algae biomass for extraction of biofuel has been disclosed. Although various embodiments of the invention have been shown and described, it should be apparent that many variations and alternative embodiments could be implemented in accordance with the teachings set forth herein. It will therefore be understood that the invention is not to be in any way limited except in accordance with the spirit of the appended claims and their equivalents.

What is claimed is:

1. A method for extracting biofuel from algae biomass using high velocity adiabatic impact compaction, comprising impacting a quantity of algae biomass with a power ram at a controlled velocity to deliver an impulse of sufficient magnitude to disrupt the algae outer cell wall structure.

2. A method in accordance with claim 1, wherein said impact has a duration of less than approximately 3 milliseconds.

3. A method in accordance with claim 1, wherein said algae biomass comprises a microalgae material, a macroalgae material, or a mixture of microalgae and macroalgae material.

4. A method in accordance with claim 1, wherein said algae biomass comprises a partially-dried algae powder.

5. A method in accordance with claim 1, wherein said power ram velocity and said impulse delivered by said power ram are selected according to a mass of said algae biomass.

6. A method in accordance with claim 1, wherein said power ram velocity ranges between approximately 3-5 meters/second to approximately 150-200 meters/second and said power ram impulse ranges between approximately 0.5-2.0 Newton-seconds to approximately 5000-12,000 Newton-seconds.

7. A method in accordance with claim 1, wherein said power ram velocity ranges between approximately 10 meters/second to approximately 50 meters/second and said power ram impulse ranges between approximately 55 Newton-seconds to approximately 280 Newton-seconds.

8. A method in accordance with claim 1, wherein said power ram delivers a specific impulse to said algae biomass of between approximately 9-24 Newton-seconds/gram of said algae biomass.

9. A method in accordance with claim 1, wherein said power ram delivers a kinetic energy to said algae biomass of between approximately 280-7100 Joules.

10. A method in accordance with claim 1, wherein said power ram delivers a specific kinetic energy to said algae biomass of between approximately 120-503 Joules/gram of said algae biomass.

11. A method in accordance with claim 1, wherein said algae biomass is compacted by said impact to a density of approximately 0.7-0.95 grams per cubic centimeter or higher.

12. A method in accordance with claim 1, wherein said algae biomass is pre-compacted prior to said impact to a density in a range of 0.1 to 0.5 grams per cubic centimeter.

13. A method in accordance with claim 1, wherein said velocity and impulse delivered by said power ram are determined according to one or more of a mass of said algae biomass, a type of said algae biomass, a water content of said algae biomass, and a pre-compaction density of said algae biomass.

14. A method in accordance with claim 1, wherein said algae biomass has a mass within a range of approximately 0.1 grams to approximately 7 kg.

15. A method in accordance with claim 1, wherein said power ram travels at a velocity of not less than approximately 8 meters/second when impacting said algae biomass.

16. A method in accordance with claim 1, wherein said method is performed with an additional power stroke applied following said impact and prior to ejection of said algae biomass.

17. A method in accordance with claim 16 wherein said additional ram power stroke has a duration of up to approximately 100 milliseconds.

18. A method in accordance with claim 1, further including one or more processing operations of pre-compaction of said algae biomass, post adiabatic impact pressing of said algae biomass, rapid pressure release post adiabatic impact and/or additional power stroke, and post adiabatic impact centrifugal processing in order to aid extraction of internal oils from said algae biomass and to separate said oils from remaining algae cell components.

19. A method for cracking the outer cell walls of algae biomass to enable the release and extraction of internal oils using high velocity adiabatic impact, comprising:
impacting a quantity of algae biomass with a power ram at a controlled velocity in multiple controlled impacts of selective intensity, each impact delivering an impulse from said power ram in a range of approximately 0.5-2.0 Newton-seconds for relatively small quantities of said algae biomass to approximately 5,000-12,000 Newton-seconds for relatively large quantities of said algae biomass, to adiabatically crack the outer walls of and compact and said algae biomass in controlled incremental stages to a final density of approximately 0.7-0.95 grams per cubic centimeter or higher.

20. A method for cracking the outer cell walls of algae biomass to enable the release and extraction of internal oils using high velocity adiabatic impact, comprising:
impacting a quantity of algae biomass with a power ram in a single impact of controlled velocity and impulse on said powder material, said impact lasting not more than approximately 3 milliseconds to adiabatically crack the outer walls of and compact said algae biomass to final density of approximately 0.7-0.95 grams per cubic centimeter or above; and
said velocity and impulse of said power ram being determined according to the mass of said algae biomass, the type of said algae biomass, the water content of said algae biomass, and the pre-compaction density of said algae biomass.
